(19) United States
(12) Patent Application Publication
(10) Pub. No.: US 2017/0273746 A1
(43) Pub. Date: Sep. 28, 2017

(54) OPTICAL SHAPE SENSING FOR SOFT TISSUE BALANCING IN ORTHOPEDICS

(71) Applicant: KONINKLIJKE PHILIPS N.V., EINDHOVEN (NL)

(72) Inventors: MOLLY LARA FLEXMAN, MELROSE, MA (US); DAVID PAUL NOONAN, NEW YORK, NY (US); ALEKSANDRA POPOVIC, BOSTON, MA (US)

(21) Appl. No.: 15/508,508

(22) PCT Filed: Sep. 8, 2015
(86) PCT No.: PCT/IB2015/056857

§ 371 Date: Mar. 3, 2017

Related U.S. Application Data
(60) Provisional application No. 62/047,330, filed on Sep. 8, 2014.

(51) Int. Cl. A61B 34/20; A61F 2/46; A61B 34/10; A61B 5/11

(52) U.S. Cl. CPC A61B 34/20; A61B 5/1128; A61F 2/4657; A61B 34/10; A61B 2034/2061; A61B 2090/373

(57) ABSTRACT

An optical shape sensing system includes an optical shape sensing fiber (102) configured to be integrated with soft tissue in a region. An optical shape sensing module (115) is configured to receive feedback from the optical shape sensing fiber and determine position and orientations of the optical shape sensing fiber. A balancing module (140) is configured to employ the position and orientation of the optical shape sensing fiber to indicate when a balance criterion is met in soft tissue as a result of adjustments in the region.

### BACKGROUND

Technical Field

This disclosure relates to medical instruments and more particularly to shape sensing optical fibers in medical applications for soft tissue balancing during computer aided procedures.

Description of the Related Art

Computer assisted surgery (CAS) systems are used for preoperative planning and intra-operative surgical navigation. In this context, preoperative planning refers to any computer assisted determination of surgical steps, such as cutting, incisions, targeting, etc. Planning can occur before or during a procedure. The preoperative planning often uses 2D or 3D images of a patient using any medical imaging modality (computed tomography (CT), magnetic resonance imaging (MRI), ultrasound, X-ray, endoscopy, etc.) or anatomical models (e.g., a knee model). In the context of CAS, surgical navigation refers to live tracking of instruments and patient anatomy enabling surgeons to precisely execute the pre-operative plan. Surgical navigation is implemented using tracking technologies.

An example of tracking technology is line-of-sight optical tracking. Line-of-sight optical tracking technology uses an optical camera either operating in the visible or infra-red range. The camera is configured to detect markers in its field of view and infer position and orientation of arrangement of markers based on their relative position. Commonly, two or more cameras arranged in a known configuration are used to enable stereo vision and depth perception. This tracking technology requires un-interrupted line-of-sight between the camera(s) and the markers.

Total knee replacement requires that portions of the femur and tibia bones be removed and replaced with implantable artificial components. CAS is used in total knee replacement to plan the appropriate cut planes using the preoperative planning module and to enable execution of the plan by tracking bone and instruments during the procedure. The bones are often resected with the use of cutting blocks that guide the cutting planes so that they are correctly positioned and angled to accept and align the artificial components to be implanted. CAS aims to improve both the position and orientation of the cutting block and of the subsequent implants to return the joint to its optimal biomechanics.

A line-of-sight optical tracked CAS system for total knee replacement involves a set of line-of-sight optical tracking attachments that are attached to the patient to provide anatomical tracking. A line-of-sight optical tracking attachment is rigidly attached to the bone through one or more screws and extends a distance away from the bone. In total knee replacement these trackers are attached to both the femur and tibia to provide the live anatomical tracking.

Existing optical CAS systems suffer from a number of disadvantages. Line-of-sight optical CAS systems require an unobstructed path between the detection cameras and the tracking attachments. Any tracking attachments that are not visible by the cameras cannot provide a valid measurement. It can be difficult to maintain an unobstructed path during all parts of the procedure, especially when, e.g., a bone is manipulated to test the dynamic biomechanics. These CAS systems not only require line-of-sight, but are also only accurate within a defined volume. This volume is with respect to the camera position and can be difficult to maintain throughout the procedure, especially during manipulation of the joint. To achieve the required accuracy, line-of-sight CAS systems typically use reflective balls arranged into optical tracking attachments which can have lengths up to 20 cm in the largest dimension. Such large attachments limit the physical workspace available to the clinicians and risk collisions intra-operatively. Due to the size and weight of the optical tracking attachments, a large screw pin is needed to rigidly and accurately attach to the bone. In some cases two screw pins are needed for a single tracking attachment. These screw pins can lead to adverse effects such as stress fractures (especially in the case of two pins used close together), infection, nerve injury, pin loosening (leading to additional pins or inaccuracies in the measurement), etc.

Electromagnetic (EM) navigation systems also suffer from a number of disadvantages. Similar to line-of-sight tracking, it can be difficult to maintain an optimal clinical workflow while also satisfying the requirements of the EM system. The EM system only provides accurate measurements within a defined volume with respect to position of the field generator. Further, metal in the EM field can generate interference and degrade the accuracy of the measurement.

Soft tissue balancing involves movement of, e.g., a knee, between flexion and extension. Following the resection of the bone, trial femoral and tibial implants are positioned to assess the size, position and orientation of the implants and the resulting joint biomechanics. The appropriate thickness of an insert is selected such that in both extension and flexion, the gap between the two bones is the same, and that medial and lateral forces applied to the spacer/ligaments are balanced. The alignment of two drill holes that will hold the insert in place is important in maintaining a smooth and balanced motion through both flexion and extension. In some cases it is necessary to release one of the ligaments (for example, by multiple needle punctures to the ligament). If the ligaments are not balanced on either side of the knee, it will cause instability, and can lead to discomfort, longer rehabilitation, and early failure of the implant.

### SUMMARY

In accordance with the present principles, an optical shape sensing system includes an optical shape sensing fiber configured to be integrated with soft tissue in a region. An optical shape sensing module is configured to receive feedback from the optical shape sensing fiber and determine position and orientations of the optical shape sensing fiber. A balancing module is configured to employ the position and orientation of the optical shape sensing fiber to indicate when a balance criterion is met in soft tissue as a result of adjustments in the region.

Another optical shape sensing system includes an optical shape sensing module configured to receive feedback from one or more optical shape sensing fibers. The one or more optical shape sensing fibers are integrated with soft tissue in a region and are configured to identify characteristics of the soft tissue. The one or more optical shape sensing fibers are configured in a pattern about the region to provide measurements in accordance with flexing of the region. The one or more optical shape sensing fibers are employed to positionally track anatomical positions in a coordinate system. A balancing module is configured to employ the position and orientation of the one or more optical shape sensing fibers to indicate when a balance criterion is met in soft tissue as a result of adjustments in the region. An anatomical image is included in the coordinate system wherein positional changes from the one or more optical shape sensing fibers are registered with the anatomical image and viewed on a display.

An implantable device for placement between bones includes a base material configured to form a substrate to be disposed between bones in a joint replacement procedure. One or more optical shape sensing fibers are configured in a pattern and integrated internally within the base material. The one or more optical shape sensing fibers are configured to measure position and orientation of placement of the implantable device between the bones.

A method for tracking soft tissue using an optical shape sensing system includes integrating one or more optical fiber sensors into soft tissue in a joint region; identifying positions of the soft tissue using the one or more optical shape sensing fibers during flexing of the joint region; testing soft tissue balancing of the joint region based upon the positions identified during the flexing; and making adjustments to the soft tissue balancing.

These and other objects, features and advantages of the present disclosure will become apparent from the following detailed description of illustrative embodiments thereof, which is to be read in connection with the accompanying drawings.

### BRIEF DESCRIPTION OF DRAWINGS

This disclosure will present in detail the following description of preferred embodiments with reference to the following figures wherein:

FIG. 1 is a block/flow diagram showing a shape sensing system for tracking soft tissue movement and forces in accordance with one embodiment;

FIG. 2 is a diagram showing a knee joint having shape sensing optical fiber sensors integrated therein by subcutaneous threading, suturing and using a needle-like element in accordance with useful embodiments;

FIG. 3 is a diagram showing a knee joint showing a flexion gap and an extension gap associated with knee flexure;

FIG. 4 is a diagram showing a knee joint having shape sensing optical fiber sensors integrated thereon using a sleeve and adhesive in accordance with useful embodiments;

FIG. 5A is a diagram showing a knee joint having a shape sensing optical fiber sensor installed in an articulated spacer in a wave pattern in accordance with one embodiment;

FIG. 5B is a diagram showing a knee joint having a shape sensing optical fiber sensor installed in an articulated spacer in a spiral pattern in accordance with another embodiment; and

FIG. 6 is a flow diagram showing a method for shape sensed tracking of soft tissue in accordance with illustrative embodiments.

### DETAILED DESCRIPTION OF EMBODIMENTS

In accordance with the present principles, systems and methods are provided for optical shape sensing that can be used for displaying relative positions of soft tissue overlaid on an anatomic map or other images during a surgical procedure. In one embodiment, the optical shape sensing fiber can be attached to or sutured in the patient or included in an instrument. The optical shape sensing measurement can be registered to the anatomical map. The position of the optical shape sensing markers with respect to an anatomic map can be displayed for a user, and the anatomical map can be dynamically updated based on the optical shape sensing information. In addition, optical shape sensing fiber may be attached to orthopedic or other instruments such as drills and cutting rigs, trial implants and final implants, etc. to track their positions as well.

In one embodiment, optical shape sensing is employed for tracking soft tissue in orthopedic procedures. Optical shape sensing systems may be attached to the ligaments, skin, inserts, etc. or combinations thereof.

Optical shape sensing systems may also be employed for force sensing of articular spacers or other regions or features. Soft tissue balancing after resection ensures that the flexion and extension gap are balanced. If the gaps are balanced, the appropriate spacer is selected, and the implants are correctly positioned and oriented, then the medial and lateral forces experienced by the ligaments should also be balanced.

The present principles address some of the following issues. Since conventional optical trackers only track the bone, they do not currently help significantly during the soft tissue balancing step of the procedure. The present principles include OSS devices that can be employed to provide relevant measurements during soft tissue balancing and provide tracking of the surface of the tissue during the procedure for protection of the ligaments. During some procedures, the forces applied on the joint can cause tearing of the ligaments. This prolongs patient recovery. It is useful to provide a way to monitor and warn against such events.

Optical shape sensing (OSS) uses light along a multicore optical fiber to reconstruct the shape along that fiber. The principle involved makes use of distributed strain measurement in the optical fiber using characteristic Rayleigh backscatter or controlled grating patterns. The shape along the optical fiber begins at a specific point along the sensor, known as the launch or z=0, and the subsequent shape position and orientation are relative to that point. The optical fiber may be, e.g., 200 microns in diameter and can be up to a few meters long while maintaining millimeter-level accuracy. Optical shape sensing fibers can be integrated into a wide range of medical devices to provide live guidance medical procedures. As an example, a guidewire or catheter may be employed for navigation to an artery with the optical shape sensing measurement overlaid upon a pre-operative image.

It should be understood that the present invention will be described in terms of medical instruments; however, the teachings of the present invention are much broader and are applicable to any fiber optic instruments. In some embodiments, the present principles are employed in tracking or analyzing complex biological or mechanical systems. In particular, the present principles are applicable to internal tracking procedures of biological systems, procedures in all areas of the body such as the lungs, gastro-intestinal tract, excretory organs, blood vessels, etc. The elements depicted in the FIGS. may be implemented in various combinations of hardware and software and provide functions which may be combined in a single element or multiple elements.

The functions of the various elements shown in the FIGS. can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Thus, for example, it will be appreciated by those skilled in the art that the block diagrams presented herein represent conceptual views of illustrative system components and/or circuitry embodying the principles of the invention. Similarly, it will be appreciated that any flow charts, flow diagrams and the like represent various processes which may be substantially represented in computer readable storage media and so executed by a computer or processor, whether or not such computer or processor is explicitly shown.

Furthermore, embodiments of the present invention can take the form of a computer program product accessible from a computer-usable or computer-readable storage medium providing program code for use by or in connection with a computer or any instruction execution system. For the purposes of this description, a computer-usable or computer readable storage medium can be any apparatus that may include, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W), Blu-Ray™ and DVD.

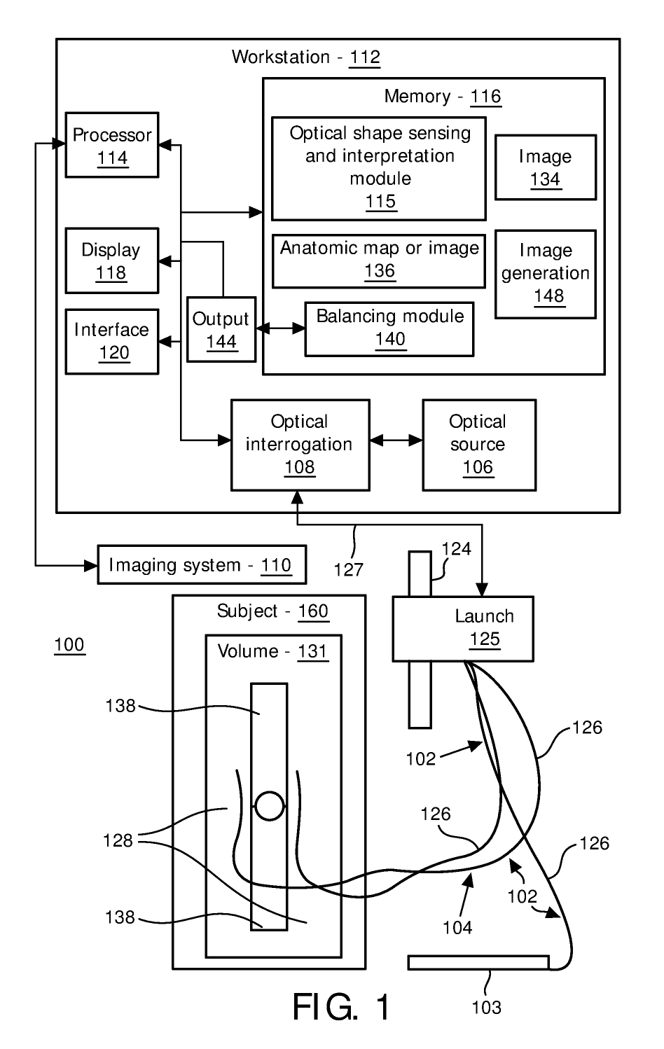

Referring now to the drawings in which like numerals represent the same or similar elements and initially to FIG. 1, a system 100 for optical shape sensing guidance in orthopedic and other applications using shape sensing enabled devices is illustratively shown in accordance with one embodiment. System 100 may include a workstation or console 112 from which a procedure is supervised and/or managed. Workstation 112 preferably includes one or more processors 114 and memory 116 for storing programs and applications. Memory 116 may store an optical shape sensing module 115 configured to interpret optical feedback signals from a shape sensing device or system 104. Optical shape sensing and interpretation module 115 is configured to use the optical signal feedback (and any other feedback, e.g., electromagnetic (EM) tracking) to reconstruct deformations, deflections and other changes associated with bones or joint positions or positions of other anatomical features, including skin, ligaments, tendons, muscles and other materials or tissues.

The shape sensing system 104 includes one or more optical fiber sensors 102. Each sensor 102 includes optical fibers 126 which are configured in a set pattern or patterns. The optical fibers 126 connect to the workstation 112 through a launch mount 125 and cabling 127 (including a communication optical fiber). The cabling 127 may include fiber optics, electrical connections, other instrumentation, etc., as needed. The cabling 127 interfaces with an optical interrogation unit 108 that may include or work with an optical source or sources 106. The interrogation unit 108 sends and receives optical signals from the shape sensing system 104. An operating room rail 124 may include the launch mount 125 that includes a reference point or launch point (z=0) for the one or more optical sensors 102.

Shape sensing system 104 with fiber optics may be based on fiber optic Bragg grating sensors. A fiber optic Bragg grating (FBG) is a short segment of optical fiber that reflects particular wavelengths of light and transmits all others. This is achieved by adding a periodic variation of the refractive index in the fiber core, which generates a wavelength-specific dielectric mirror. A fiber Bragg grating can therefore be used as an inline optical filter to block certain wavelengths, or as a wavelength-specific reflector.

Inherent backscatter in conventional optical fiber can be exploited for optical shape sensing (OSS). One such approach uses Rayleigh scatter (or other scattering) in standard single-mode communications fiber. Rayleigh scatter occurs as a result of random fluctuations of the index of refraction in the fiber core. These random fluctuations can be modeled as a Bragg grating with a random variation of amplitude and phase along the grating length. By using this effect in three or more cores running within a single length of multicore fiber, the 3D shape and dynamics of the surface of interest can be followed.

Fiber Bragg Gratings (FBGs) may also be employed for OSS, which use Fresnel reflection at each of the interfaces where the refractive index is changing. For some wavelengths, the reflected light of the various periods is in phase so that constructive interference exists for reflection and, consequently, destructive interference for transmission. The Bragg wavelength is sensitive to strain as well as to temperature. This means that Bragg gratings can be used as sensing elements in fiber optical sensors. In an FBG sensor, the measurand (e.g., strain) causes a shift in the Bragg wavelength.

One advantage of OSS is that various sensor elements can be distributed over the length of a fiber. Incorporating three or more cores with various sensors (gauges) along the length of a fiber that is embedded in a structure permits a three-dimensional form of such a structure to be precisely determined, typically with millimeter-scale accuracy. Along the length of the fiber, at various positions, a multitude of FBG sensors can be located (e.g., 3 or more fiber sensing cores). From the strain measurement of each FBG, the curvature of the structure can be inferred at that position. From the multitude of measured positions, the total three-dimensional form is determined.

In one embodiment, the one or more optical sensors 102 are connected through soft tissue 128 or other anatomical features by suturing or otherwise attaching the optical sensors 102 through or on the soft tissue 128. The optical sensors 102 may include a plurality of different configurations and pass through different materials. The optical sensors 102 may be oriented around bones, through or between joints or other soft tissue or applied to skin or other exposed tissue. The one or more optical sensors 102 may also be connected to a medical device 103, either directly or as part of a sleeve, insert, housing, tube or other fitting. The medical device may be, for example, a catheter, a guidewire, a probe, an endoscope, a robot, an electrode, a filter device, a balloon device, a drill, a cutting rig, a pointer, an implant, or other medical component, etc.

The optical fiber sensors 102 are configured to be flexible and have a small outer diameter to ensure flexibility and to reduce soft tissue trauma. The optical fiber sensors 102 may be coated or sheathed in a material suitable for use within the body. For example, the optical fiber sensors 102 may include a hypotube, coiled tube, pebax extrusion, any other biocompatible coating or tubing.

Workstation 112 includes an image generation module 148 configured to receive feedback from the shape sensing system 104 and record position data as to where the one or more optical sensors 102 have been within a volume 131. An image 134 of the one or more optical sensors 102 within the space or volume 131 can be displayed on a display device 118. Workstation 112 includes the display 118 for viewing internal images of a subject (patient) or volume 131 and may include the image 134 as an overlay or other rendering of the sensing device 104 on images collected by an imaging device 110. The imaging device 110 may include any imaging system (e.g., CT, ultrasound, fluoroscopy, MRI. etc.). Display 118 may also permit a user to interact with the workstation 112 and its components and functions, or any other element within the system 100. This is further facilitated by an interface 120 which may include a keyboard, mouse, a joystick, a haptic device, or any other peripheral or control to permit user feedback from and interaction with the workstation 112.

Workstation 112 includes a balancing module 140 configured to receive feedback from the shape sensing system 104 and make decisions and comparisons (balances) involving soft tissues, implant sizing, force or displacement balancing, joint evaluation and the like. The balancing module 140 receives information from the soft tissue optical sensors 102 within the volume 131. The position and orientation data from the fiber sensors 102 is employed to compare changes in position of soft tissue, to compare changes in gaps or other dimensions during flexing of a joint or muscle, etc. The changes may be compared to acceptable tolerances or previously measured criteria. For example, the sensors 102 can be employed to provide relevant measurements during soft tissue balancing and provide tracking of the surface of the tissue during the procedure for protection of the ligaments or other tissues. During some procedures, the forces applied on the joint can cause tearing of the ligaments. This prolongs patient recovery. It is useful to provide a way to monitor and warn against such events. The balancing module 140 tracks this information and outputs useful information, warnings and alerts to an operator in real-time. The balancing module 140 is configured to employ the position and orientation of one or more optical shape sensing fibers 102 to indicate when a balance criterion is met in soft tissue as a result of adjustments in the region. The balancing module 140 may be configured to provide measurements and feedback for a plurality of tasks. Some examples follow.

In one embodiment, the balancing module 140 receives input from a sensor 102 for a ligament to identify when the ligament is being stretched too much and to warn the operator by an output device 144 (e.g., sound (speaker), visual feedback (light, display, etc.), haptic feedback, etc.). The balancing criterion includes an acceptable amount of stretch versus the measured stretch. In another embodiment, two ligaments may be tracked by sensors 102 to identify when the forces on both ligaments are within a force threshold (balance criteria) during flexion and extension. A balance can be indicated to the operator on output device 144. In another embodiment, two ligaments may be tracked by sensors 102 to confirm that the shape profile of the ligaments is parallel (balance criteria) during flexion and extension. Confirmation can be indicated to the operator on output device 144. An angular metric or plot may also be provided as to the angle between the two ligaments during flexion and extension.

In other embodiments, input from a skin surface around the knee that is tracked by sensors 102 may be overlaid on a shape profile (balance criteria) of the skin around the knee during flexion and extension on top of a profile from prior to the operation. This information may be captured throughout the procedure. The same measurements may be performed periodically during rehabilitation. An indication of similarity can be indicated to the operator on the output device 144.

Information from ligaments and/or skin surface may be collected during testing of a trial implant. The measured shape and force information from sensors 102 may be employed to either accept a current implant, or predict a correct implant that should be used. In addition, shape information may be collected from the trial implant, which may include its own sensor(s) 102. Based on collected information from the bones, ligaments, skin, etc., the user is informed through output device 144 (which may include the display 118) how the implant should be rotated to fix any misalignment (1 degree clockwise, for example). When the user then adjusts the implant, the balancing module rechecks the position and confirms visually (through output device 144) that the adjustment brought the implant to its intended position. When the actual implant (final implant) is put in, its position and orientation are shape sensed with sensors 102, and the balancing module 140 confirms through the output device 144 to the user that the position and orientation match the trial position and orientation. It should be understood that other measurements, comparisons, checks, etc. may be performed using the balancing module 140, in addition to or instead of those described herein.

The system 100 is based on optical shape sensing and can be used for displaying the deformations of soft tissue and the relative position of bones overlaid on an anatomic map 136 (e.g., an anatomical image of volume 131) during a surgical procedure or otherwise. The system 100 includes the integration of the optical shape sensing fibers 102 into soft tissue of a patient 160 (e.g., the skin, muscle, ligaments, etc.), registration of the optical shape sensing instruments 102 to the anatomical map 136, display of the position(s) of the optical shape sensing devices 102 with respect to each other and the anatomic map 136, attachment of optical shape sensing fiber 126 to orthopedic instruments 103 such as drills, cutting rigs, etc.

The optical sensors 102 may have their coordinate systems registered to bone positions, a global coordinate system or any other coordinate system. Optical shape sensing fibers 102 can be registered to each other using multiple techniques including shape-to-shape registration, mechanical registration of launch positions, point-based registration, etc. To make the shape sensing measurements useful to the clinician, the measurements need to be provided in the context of an anatomic map 136. The anatomical map is preferably a pre-operative image (such as, a CT image or MRI). In some cases, an anatomical model is morphed to match the feature measurements during a registration step. Herein, a 3D surface or volume of the bone or other feature, acquired preoperatively or from any source, will be referred to as a model.

Once the optical shape sensing fiber 102 is placed and registered to the anatomical map 136 or other reference (e.g., bone 138), the fiber positions can be displayed to the operator (e.g., on display 118). The display of OSS data on an anatomical map may take many forms and provide a plurality of functions.

The present principles apply to any use of an optical shape sensing fiber for surgical guidance and navigation. In particularly useful embodiments, the present principles may be employed in knee replacement surgery, anterior cruciate ligament (ACL) repair, hip replacement, brain surgery, spinal surgery, elbow surgery and other such applications. In addition, the OSS may employ any type of reflective or scattering phenomena such as, e.g., Rayleigh scatter (enhanced and regular) as well as Fiber Bragg implementations of shape sensing fiber. The present principles may be employed with manual and robotic systems.

The optical shape sensing tracking in accordance with the present principles can be employed to provide pre-procedural planning, including implant sizing, etc., understand the biomechanics of a joint including the range of flexion and extension and identification of any misalignment between the bones that may lead to balance issues, instability, etc. This is done through OSS tracking in various positions with the resulting biomechanics and alignment features being visualized virtually and displayed to the operator using the balancing module 140. Intra-procedural planning and post-procedural evaluation of the joint biomechanics may also be provided by the balancing module 140.

The fiber optic sensors 102, in accordance with the present principles, may be employed for a plurality of different functions. In one embodiment, the fiber optic sensors 102 are employed for ligament tracking. The ligaments may be tracked during a procedure. Tracking the soft tissue using the balancing module 140 provides a safety mechanism to warn a doctor using the output device 144 when there is an unusual position or force applied to the soft tissue. This could help to prevent tearing of the ligaments during the procedure, but there are many other uses for the present principles.

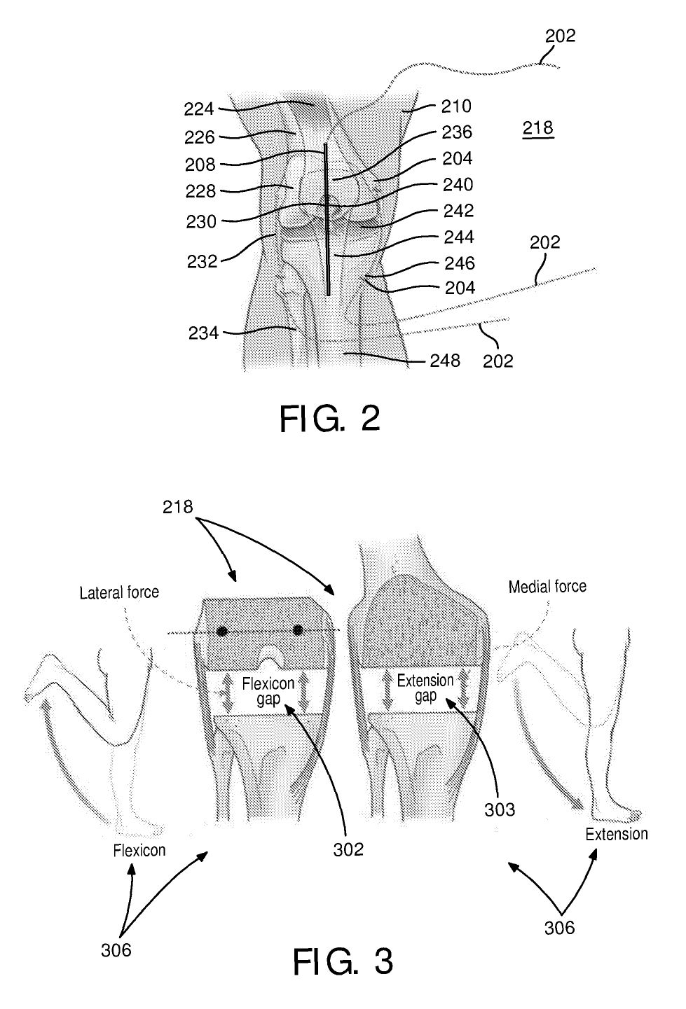

Referring to FIG. 2, in one embodiment, an optical fiber sensor 202 (or a device with the optical fiber embedded therein) is threaded through soft tissue (e.g., through a ligament or tissues surrounding the ligament). In another embodiment, the optical fiber sensors 202 are sutured by sutures 204 at two or more points close to a bone attachment or other reference point. In another embodiment, the optical fiber sensor 202 can be embedded in or otherwise attached to a flexible needle 208 (or similar flexible elongated tool or instrument), and the needle 208 can be inserted through skin 210 (or other tissue) and tunneled along the surface of a ligament, etc.

FIG. 2 shows an anatomical diagram of a knee joint 218. The diagram includes quadriceps muscle 224, femur 226, articular cartilage 228, anterior cruciate ligament (ACL) 230, lateral collateral ligament (LCL) 232, fibula 234, patella (knee cap) 236, posterior cruciate ligament (PCL) 240, meniscus 242, patellar ligament 244, medial collateral ligament (MCL) 246 and tibia 248.

By embedding the optical fiber sensor 202 or sensors in soft tissues of the knee joint 218 (either directly or as incorporated in or attached to an embedded material or sleeve), the ability to track not only the position and twist of the knee joint 218 and its components, but also the strain on ligaments 230, 232, 240, 244, 246 or other soft tissue during a procedure, is achieved. Force measured across medial ligaments 246 and lateral ligaments 232 can be used to align inserts placed in gaps 302, 303 (FIG. 3) and select implant sizes. This can be measured dynamically during flexion 306 and extension 304 of a knee joint 218 as depicted in FIG. 3. Existing solutions typically only perform this measurement at two fixed positions. In accordance with present principles, extension 304 and flexion 306 measurements could be performed continuously during motion from extension 304 to flexion 306 using OSS.

Tracking the soft tissue, in addition to tracking the positions of the bones (using other tracking methods), during the extension 304 and flexion 306 of the joint 218 can provide a more complete kinematic model than using the positioning of the bones (e.g., femur 226 and tibia 248) alone. The optical fiber sensors 202 may remain in the joint during and after rehabilitation. Measurements during the procedure may be correlated with post-procedural measurements during rehabilitation. In a further embodiment, two sets of measurements can be recorded. A first measurement of soft tissue deformation is recorded before incisions on the knee bones are done. A second measurement is continuously performed during selection of appropriate cuts or instruments, such as spacers. These measurements are performed with the same fiber or a different set of fibers registered at the launch fixture, thus, the sensors 102 can be visualized in the same coordinate frame. Before incision, the surgeon can choose an area around preoperative soft tissue deformations where post-operative deformations would be optimal. The visualization can show the post-incision shape within this volume and notify surgeon if the deformation exceeds desired levels.

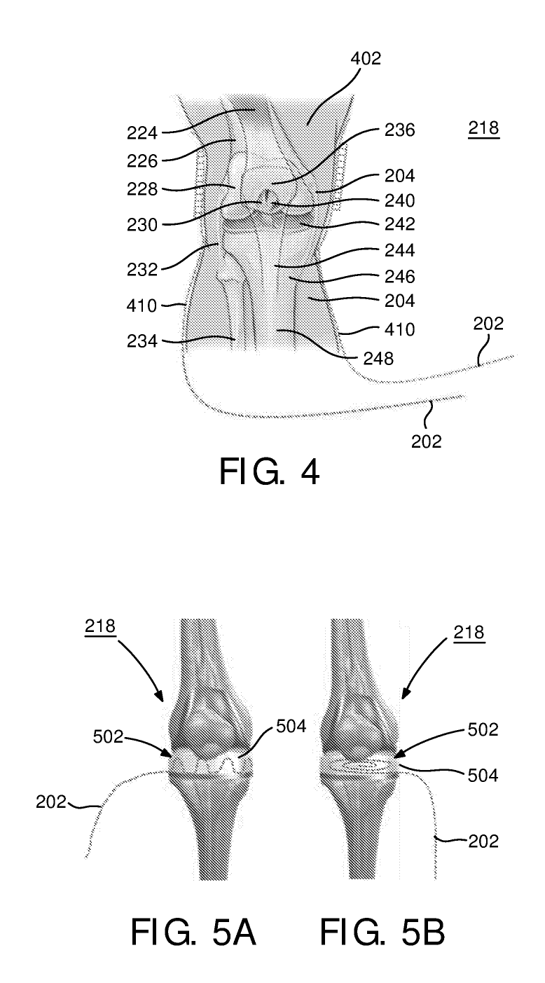

Referring to FIG. 4, while ligament tracking employs a semi-invasive attachment of optical fiber sensors 202 onto or into the ligament, attaching the optical fiber sensor 202 to skin 402 of the patient is less invasive, but also provides a less direct measurement. Tracking of the skin 402 around the knee joint 218 can be performed in multiple ways. In one embodiment, a sleeve 404 may be positioned around the knee joint 218 (FIG. 2) with optical shape sensing fibers 202 embedded within, attached to or otherwise incorporated in the sleeve 404. In this embodiment, the fiber(s) 202 can be embedded, attached or incorporated in multiple different ways. Examples include fiber(s) 202 running parallel to a longitudinal axis of a leg on either side, fiber(s) 202 taking ‘wave’ like paths (e.g., sinusoidal patterns) along the longitudinal axis of the leg, or wrapping around the knee joint 218 in a helix shape (as permitted by the surgical incision). A sleeve, as the term is used herein, may be a sheath, an insert, housing, tube or other fitting in which optical fibers are embedded, encapsulated, contained or otherwise held,

In one embodiment, the sleeve 404 may be placed over the joint 218 and the optical fiber sensors 202 may be threaded under or into the sleeve 402 when the sleeve 402 is in place. In this way, a pattern may be selected for the pattern of the optical fiber sensors 202 in real-time.

Skin attachments of the optical fiber sensors may be applied using an adhesive 410 or the like, which mount the optical fiber sensors 202 on or around the knee joint 218, e.g., on either side of the knee joint 218 as illustratively depicted in FIG. 4. The adhesive 410 may be applied to the skin around the joint and the optical fiber sensors 202 may be disposed in the adhesive in any desired pattern or patterns, e.g., waves, helix, longitudinal stripes, etc.

Referring to FIGS. 5A and 5B, two illustrative configurations are depicted for monitoring force in an implantable device 502, such as, e.g., an articular spacer, insert, implant, etc. Force sensing of the device 502 is provided to measure medial and lateral forces for soft tissue balancing. As depicted in FIG. 3, gaps due to flexion and extension need to be balanced. During knee replacement surgery, force sensing in an insert provides a strong indicator of soft tissue balancing. There are existing conventional devices that use the force sensed by an articular spacer during flexion and extension to balance the tissue (for example, a commercially available e-LIBRA™ device). The doctor implants a spacer enabled with conventional force sensors and then uses the readout to adjust the positioning of the implants until the sensor sees balanced forces on the medial and lateral part of the spacer. One limitation of the e-LIBRA™ device is that it only provides two uniaxial force measurements—one on the medial side and one on the lateral side(s). Another limitation of the e-LIBRA™ device is that it does not have any knowledge of the spatial position of the force measurements within the device. Alternatively or additionally, force sensing can be done using the optical fiber.

In accordance with the present principles, the device 502 is configured for placement between bones. The device 502 includes a base material 504 to form a substrate configured for surgical use as a spacer, etc. to be disposed between bones in a joint replacement procedure or the like. The base material 504 may include any material suitable for use inside the body of a living being and preferably includes a resilient and compliant material. The device 502 includes one or more embedded shape sensing fibers 202 that are arranged within the spacer in a pattern and integrated internally within the base material 504 to measure position and orientation of placement of the implantable device 502 between bones. The shape sensing fiber(s) 202 may be in an insert or attachment to the device 502 or base material 504. The shape sensing fiber 202 can be used in combination with embedded force sensors (not shown) to have spatially-resolved force measurements within the device 502. The optical shape sensing fibers 102 can be employed to measure a force distribution within the device 502 as well by, for example, measuring strain in the central core of the fiber (e.g., instead of or in addition to independent force sensors).

The shape sensing fiber itself can be configured to provide spatially-resolved force measurements in 3-axes (x, y and z) through its integration into the device 502. Such an implementation provides highly spatially resolved force measurements within the spacer and improves the ability of the clinician to fine tune the implant to balance the knee for all axes of movement. The OSS fiber 202 may be arranged in any number of patterns.

The optical shape sensing fiber 202 may be configured in a pattern and integrated internally within the base material 504. The pattern may include at least one of a sinusoid, a spiral, a helix, a loop, a mesh, etc. The spacer is used with trial implants, e.g., for the femur and tibia. These trial implants can also be shape sensed (as with device 502) to monitor position and force information during the soft tissue balancing. Shape sensed tracking of the final implants can be used to make sure they are positioned as determined by the trial implants and to predict any errors introduced during a final stage of the procedure. The shape sensing fiber 202 could be removed before finalizing the procedure, or left embedded in the implant (and may be employed for post-procedure testing and measurements).

In FIG. 5A, the OSS fiber 202 is arranged in a sinusoidal wave pattern. In FIG. 5B, the OSS fiber 202 is arranged in a spiral or helical pattern. Other patterns are contemplated as well, e.g., a wave pattern in the plane of the joint between the bones, a cylindrical spiral, a loop, etc. It should be understood that the OSS fiber 202 may be removable or may remain permanently as part of the device 502. In one embodiment, the OSS fiber 202 may be employed during rehabilitation to makes force or position measurements for comparison to other measurements to determine progress or other metrics.

It should be understood that the embodiments described herein illustratively employ a knee joint. However, any joint or other anatomical feature, prosthetic or model may employ the present principles. In addition, the embodiments described herein may be combined to further increase the advantages of the present principles. For example, the sutured OSS fiber embodiments may be combined with the skin-attached OSS fiber and/or the sleeve/needle with integrated OSS fiber.

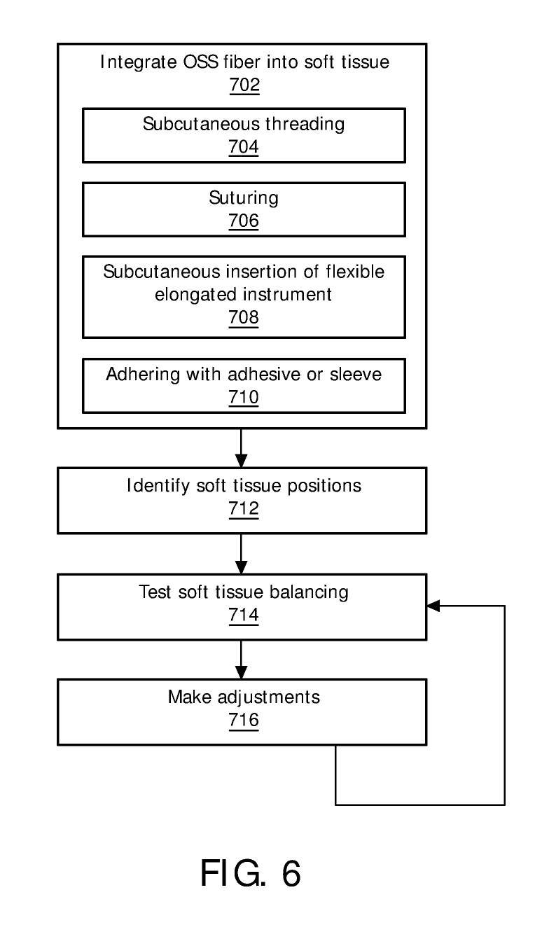

Referring to FIG. 6, a method for tracking soft tissue using an optical shape sensing system is illustratively shown. It should be understood that the present principles may be applied to tissue inside the body, to anatomical models or exterior tissue, to prosthetic limbs, to mechanical components or linkages, etc. In block 702, one or more optical fiber sensors are integrated into soft tissue in a joint or other region. In block 704, integrating the one or more optical fiber sensors may include subcutaneously threading the one or more optical fiber sensors into the soft tissue. In this embodiment, the optical fibers are inserted into the skin, muscle, ligaments, etc. This may be performed using a long suturing needle or the like to pass through large portions of the soft tissue. In block 706, integrating the one or more optical fiber sensors may include suturing the one or more optical fiber sensors to the soft tissue. In this embodiment, the optical fiber is intermittently sutured or stapled to soft tissue at different locations along its length.

In block 708, integrating the one or more optical fiber sensors may include subcutaneously inserting a flexible elongated instrument including the one or more optical fiber sensors into the soft tissue. Here, the optical fiber is disposed in a needle-like element. The needle-like element is passed into the soft tissue and remains in the soft tissue during flexing of the joint. In block 710, integrating the one or more optical fiber sensors may include adhering the one or more optical fiber sensors to the soft tissue using one or more of an adhesive or a sleeve. Adhesives may be employed to hold different patterns of optical fiber in place on the skin (or other tissue). A sleeve may be employed over the optical fibers to hold the optical fibers in place. The sleeve may also have optical shape sensing fibers integrated therein.

In block 712, positions of the soft tissue are identified using the optical shape sensing fiber during flexing of the joint or other region. Deflections, strain, forces, etc. can be determined during the flexing (e.g., flexion and extension) of the joint region. In block 714, soft tissue balancing of the joint region is tested based upon the positions identified during the flexing. Testing may uncover issues, such as, e.g., insufficient flexion gap or extension gap in knee replacement surgery, excessive force in one lateral ligament, excessive displacement on one side of the joint, etc. Testing may include, e.g., employing an insert spacer or implant having OSS fibers integrated therein. In block 716, adjustments to the soft tissue balancing are made. These may include actions to remedy insufficient flexion gap or extension gaps in knee replacement surgery, excessive force in one lateral ligament, excessive displacement on one side of the joint, etc.

Feedback is provided as to whether balancing criteria has been achieved. If the criteria is not achieved the procedure returns to block 714 and repeats until the balancing criteria has been achieved (e.g., force is balanced, displacement is balanced, gap is balances, etc.).

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function; and
e) no specific sequence of acts is intended to be required unless specifically indicated.

Having described preferred embodiments for optical shape sensing for soft tissue balancing in orthopedics (which are intended to be illustrative and not limiting), it is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims. Having thus described the details and particularity required by the patent laws, what is claimed and desired protected by Letters Patent is set forth in the appended claims.

1. An optical shape sensing system, comprising:
an optical shape sensing fiber configured to be integrated with soft tissue in a region;
an optical shape sensing module configured to receive feedback from the optical shape sensing fiber and determine position and orientations of the optical shape sensing fiber; and
a balancing module configured to employ the position and orientation of the optical shape sensing fiber to indicate when a balance criterion is met in soft tissue as a result of adjustments in the region.

2. The system as recited in claim 1, wherein the optical shape sensing fiber is threaded subcutaneously into the soft tissue to measure changes due to motion of the region.

3. The system as recited in claim 1, wherein the optical shape sensing fiber is sutured into the soft tissue to measure changes due to motion of the region.

4. The system as recited in claim 1, further comprising a flexible elongated instrument including the optical shape sensing fiber therein and configured to be subcutaneously inserted into the soft tissue.

5. The system as recited in claim 1, wherein the optical shape sensing fiber is adhered to the soft tissue.

6. The system as recited in claim 5, further comprising a sleeve configured to adhere the optical shape sensing fiber to the soft tissue.

7. The system as recited in claim 1, further comprising an implantable device configured to employ an optical shape sensing fiber to sense one or more of position, orientation and a force of the device.

8. An optical shape sensing system, comprising:
an optical shape sensing module configured to receive feedback from one or more optical shape sensing fibers, the one or more optical shape sensing fibers being integrated with soft tissue in a region and being configured to identify characteristics of the soft tissue, the one or more optical shape sensing fibers being configured in a pattern about the region to provide measurements in accordance with flexing of the region, the one or more optical shape sensing fibers being configured to positionally track anatomical positions in a coordinate system;
a balancing module configured to utilize the position and orientation of the one or more optical shape sensing fibers to indicate when a balance criterion is met in soft tissue as a result of adjustments in the region; and
an anatomical image included in the coordinate system wherein the system is configured to register positional changes from the one or more optical shape sensing fibers with the anatomical image and show the changes on a display.

9. The system as recited in claim 8, wherein the optical shape sensing fiber is threaded subcutaneously into the soft tissue to measure changes due to motion of the region.

10. The system as recited in claim 8, wherein the optical shape sensing fiber is sutured into the soft tissue to measure changes due to motion of the region.

11. The system as recited in claim 8, further comprising a flexible elongated instrument including the optical shape sensing fiber therein and configured to be subcutaneously inserted into the soft tissue.

12. The system as recited in claim 8, wherein the optical shape sensing fiber is adhered to the soft tissue.

13. The system as recited in claim 12, further comprising a sleeve configured to adhere the optical shape sensing fiber to the soft tissue.

14. (canceled)

15. An implantable device for placement between bones, comprising:
a base material configured to form a substrate to be disposed between bones in a joint replacement procedure; and
one or more optical shape sensing fibers configured in a pattern and integrated internally within the base material, the one or more optical shape sensing fiber being configured to measure position and orientation of placement of the implantable device between the bones.

16. (canceled)

17. (canceled)

18. (canceled)

19. (canceled)

20. (canceled)

21. A method for tracking soft tissue using an optical shape sensing system, comprising:
integrating one or more optical fiber sensors into soft tissue in a joint region;
identifying positions of the soft tissue using the one or more optical shape sensing fibers during flexing of the joint region;
testing soft tissue balancing of the joint region based upon the positions identified during the flexing; and
making adjustments to the soft tissue balancing.

22. (canceled)

23. (canceled)

24. (canceled)

25. (canceled)
